(19) United States
(12) Patent Application Publication
(10) Pub. No.: US 2010/0284926 A1
(43) Pub. Date: Nov. 11, 2010

(54) USE OF G-CSF FOR THE TREATMENT OF STROKE

(75) Inventors: Armin Schneider, Heidelberg (DE); Rico Laage, Schriesheim (DE); Gerhard Vogt, Heidelberg (DE); Winfried Koch, Schwetzingen (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: Sygnis Bioscience GmbH & Co. KG, Heidelberg (DE)

(21) Appl. No.: 12/677,681

(22) PCT Filed: Sep. 12, 2008
(86) PCT No.: PCT/EP08/62112

§ 371 Date: May 25, 2010

(30) Foreign Application Priority Data
Sep. 13, 2007 (EP) 07018029.4

(51) Int. Cl. A61K 38/19; A61K 49/00; A61P 25/00

(52) U.S. Cl. 424/9.2; 424/85.1

(57) ABSTRACT

The invention relates to the use of G-CSF for the treatment of cerebral stroke, particularly cerebral stroke with a large baseline infarct volume. Administration of G-CSF at a total dose of 30 to 180 μg per kg body weight over a treatment period of about 3 days is suitable for the treatment of stroke. Treatment in accordance with the invention with a total dose of 80 to 150 μg per kg body weight is preferred.

The invention relates to the use of granulocyte-colony stimulating factor (G-CSF) for the production of a medicinal product for the treatment of cerebral stroke in humans, the G-CSF being administered at a daily dose of 30 to 180 μg per kg body weight over a period of at least 2 days.

Cerebral stroke is the third most common cause of death and the main cause of dependency on care in the world. It thus represents an enormous socio-ecological burden. The aetiology of stroke is either ischaemic—as in the majority of cases—or haemorrhagic. Ischaemic stroke is usually caused by an embolus or a thrombus. As yet, there is no effective method of treating the majority of stroke patients. The only drugs so far registered for clinical use are tissue plasminogen activator (tPA) and acetylsalicylic acid. After a massive cell death in the immediate core of the infarct caused by glucose and oxygen deficiency (cerebral ischaemia), the zone of infarction grows for a few days due to secondary mechanisms such as glutamate excitotoxicity, inflammatory mechanisms, the production of free radicals and apoptotic mechanisms (Leker & Shohami, Brain Res. Rev. 2002; 39: 55-73). The infarct volume can be determined by magnetic resonance tomography; the DWI (diffusion-weighted image) method is used initially to determine the zone of previous cellular damage or destruction, whereas the PWI (perfusion-weighted image) method, which investigates the distribution of a contrast agent, provides information about the size of the tissue zone which is underperfused at the time. The tissue zone determined by PWI is often larger than that determined by DWI. In these cases, it is assumed that the restoration of blood flow (e.g. by thrombolytic treatment with tPA) can maintain the function of the part of the tissue zone determined by PWI which does not overlap with that determined by DWI, whereas it may be possible only less effectively to save the tissue area measured by DWI (Beaulieu et al. Ann Neurol 1999; 46: 568-578; Wu et al. Stroke 2001; 32: 933-942).

Most of the drugs registered so far can achieve the restoration of blood flow after an ischaemic stroke, if treatment is given promptly (no more than about 3 to 6 h after the stroke). These drugs do not have a protective effect on the neurons affected by the stroke (neuroprotection) and certainly do not have the necessary properties to encourage the formation of new neurons in the area affected (neuroregeneration). Accordingly, there is a great need for new, in particular neuroprotective and/or neuroregenerative, methods of treatment to improve the clinical outcome of cerebral stroke. Such new methods of treatment should preferably also be suitable for the delayed onset of treatment of stroke, e.g. when treatment only starts after 6 h or more.

Rating scales such as the modified Rankin scale or the NIH stroke scale (NIHSS) are generally used for the quantitative evaluation of the severity of a stroke, whether acute or under treatment. While the Rankin scale permits very rough classification of a patient's neurological status (from the value “0” for “free from symptoms” to the value “6” for “dead”), the NIH stroke scale permits a high-resolution evaluation of a patient's neurological status. To obtain the finding on the NIH stroke scale, various neurological aspects are investigated and assigned point scores. The total point score is a measure of the severity of the symptoms of stroke, the point score increasing with the severity of the symptoms. These rating scales are also suitable for monitoring the course of the symptoms after stroke and for quantifying the success of any treatment used. In general, it is possible to establish a correlation between the size of the infarct and the severity of the stroke as quantified by the stroke scale (Beaulieu et al. Ann Neural 1999; 46: 568-578). Hence, the course of the size of the infarct under treatment is also suitable for the assessment of a treatment effect.

G-CSF is a member of the group of colony-stimulating factors (CSF). These are regulatory proteins responsible for the control of the proliferation and differentiation of haematopoietic cells such as granulocytes, megakaryocytes and monocytes and also of macrophages. Without appropriate CSFs, these haematopoietic cells cannot survive or proliferate in culture. CSFs belong to the cytokine group. Together with erythropoietin (EPO) and some interleukins, they form the group of haematopoietic growth factors.

In particular, the group of CSFs includes the factors M-CSF (macrophage colony-stimulating factor; also called CSF-1), GM-CSF (granulocyte/macrophage colony-stimulating factor; also called CSF-2), G-CSF (granulocyte colony-stimulating factor; also called CSF-3) and multi-CSF (multifunctional colony-stimulating factor; also called IL3), named according to their specificity in relation to various haematopoietic cells. The purification and cloning of the individual CSFs has permitted molecular characterization. The four CSFs cited are glycoproteins, although they do not display any homology at the primary structure level (amino acid sequence) (Metcalf, Cancer 1990; 65: 2185-2194; Pimentel, Ann. Clin. Lab. Sc. 1990; 20: 36-55).

G-CSF is secreted by activated monocytes, macrophages and neutrophils, by stromal cells, fibroblasts and endothelial cells, as well as by various tumour cell lines (e.g. human bladder cancer cell line). Mature human G-CSF is a monomeric glycoprotein containing 174 amino acids, the sugar portion of which is not necessary for its biological activity. Another variant containing 177 amino acids, obtained by variant splicing of the RNA, has substantially reduced biological activity (Nagata, BioEssays 1989; 10: 113-117). G-CSF promotes the proliferation and differentiation of haematopoietic precursor cells to form neutrophilic granulocytes, which it also activates. Furthermore, G-CSF also acts as a mitogen.

On the basis of this promotion of proliferation, differentiation and activation of cells of the haematopoietic system, G-CSF is registered for the treatment of neutropenia, e.g. as a result of chemotherapy and/or radiotherapy. In addition, G-CSF is used clinically to stimulate the production of neutrophils in the bone marrow, e.g. in advance of a bone-marrow donation for bone-marrow transplantation. For a few years, G-CSF has also been approved for the treatment of neutropenia within the framework if HIV infection. Recombinant G-CSF (e.g. filgrastim, Neupogen®) is primarily used in therapy. For the treatment of neutropenia within the framework of chemotherapy and/or radiotherapy, the usual daily doses are about 5 μg (corresponding to 0.5 MIU) per kg body weight. The dose is usually administered as a subcutaneous bolus injection, as continuous subcutaneous injection, as a short-term intravenous injection (over 15 to 30 min) or as a continuous intravenous injection. In the treatment of neutropenia within the framework of HIV infection, the dose used is generally substantially lower than that used within the framework of chemotherapy and/or radiotherapy.

Recent research shows that G-CSF, in addition to its leucocyte-stimulating effect, also has other clinically relevant properties. For example, the use of G-CSF and other CSFs for the treatment of infections (WO 88/00832), for the promotion of wound healing (WO 92/14480) and for the stimulation of angiogenesis (WO 97/14307) has been described. Furthermore, Takashi and Yoshihiro reported that G-CSF and other factors are suitable for activating acetylcholine transferase (ChAT) and thus can contribute to increasing the survival times of the affected cells in various neurodegenerative diseases (e.g. Alzheimer's disease and dementia) (JP 03537151).

Buschmann and Scharper reported that G-CSF and GM-CSF have arteriogenic effects, that is, they increase the growth of collateral arteries from already existing arteriolar connections (EP 1019082). In this way, these factors can contribute to improving the restoration of blood flow of ischaemic tissue, inter alia in cerebral stroke (Buschmann et al. Circulation 2003; 108: 610-615). It is also reported that G-CSF, by stimulating the mobilization of bone-marrow stem cells, is suitable to promote the neuroregeneration of damaged nerve tissue after stroke or other neurodegenerative diseases (WO 02/099081, EP 1465653).

The recent observation that G-CSF receptors can also be found on neurons (DE 10033219) suggests that G-CSF can also have a direct effect on these cells of the CNS. In agreement with this, G-CSF has recently been shown to have a neuroprotective and neuroregenerative effect in an animal model for the treatment of focal cerebral ischaemia (Schabitz et al. Stroke 2003; 34: 745-751; Schneider et al. J Clin Invest 2005; 115: 2083-2098; WO 2004/58287; WO 2006/08582).

The object of the present invention is to make available G-CSF as a medicinal product for the treatment of stroke patients at a dose, in a dose regime and in a pharmaceutical form which has particularly appropriate efficacy without leading to adverse side effects.

Accordingly, the invention described here relates to the use of G-CSF for the production of a medicinal product for the treatment of stroke in human patients, the G-CSF being administered to the patient at a total dose of about 30 to 180 μg per kg body weight over a period of 2 to 7 days.

The invention also relates to G-CSF for use in a method for the treatment of stroke in human patients, the G-CSF being administered to the patient at a total dose of about 30 to 180 μg per kg body weight over a period of 2 to 7 days.

The invention also relates to a method for the treatment of stroke in human patients, G-CSF being administered to the patient at a total dose of about 30 to 180 μg per kg body weight over a period of 2 to 7 days.

In the context of the present invention the terms “patient” and “patients” are used interchangeably and cover both the singular and the plural. In addition, the terms “patient” and “stroke patient” are used interchangeably.

Total dose levels of 90 μg and especially 135 μg of G-CSF per kg body weight are particularly suitable for the treatment of stroke patients, as the study evaluation described in Example 1 unexpectedly shows. A further increase in the total dose above a dose level of 135 μg per kg body weight does not improve the treatment outcome. According to this study evaluation, the higher dose level of 180 μg per kg body weight as a total dose actually led to a lower treatment success in patients with a mean baseline infarct size of about 25 cm³ (DWI measurement) than the dose level of 135 μg per kg body weight (FIG. 3). According to the study evaluation, lower dosages, such as those used e.g. in the treatment of neutropenia (30 μg per kg body weight or less) led to no or to only suboptimal treatment success (FIGS. 2 and 3). Because the weight of the patients is only estimated during the treatment of stroke, it can be assumed that the total dose actually administered varies around the intended value of the dose level with a tolerance of about 10%. Accordingly, the dose level of 90 μg per kg body weight corresponds to an actually administered dose of 80 to 100 μg per kg body weight and the dose level of 135 μg per kg body weight to an actually administered dose of 120 to 150 μg per kg body weight.

It is therefore preferable to use a total dose of 80 to 150 μg of G-CSF per kg body weight (corresponding to the dose levels of 90 μg of G-CSF per kg body weight as a total dose to 135 μg of G-CSF per kg body weight as a total dose with a tolerance of about 10% in relation to the actual total dose per kg body weight administered), to be administered intravenously over a period of 3 days.

Furthermore, it is particularly preferable to use a total dose of 120 to 150 μg of G-CSF per kg body weight (corresponding to the dose level of 135 μg of G-CSF per kg body weight as a total dose with a tolerance of about 10% in terms of the actual total dose per kg body weight administered), to be administered intravenously over a period of 3 days. It is especially preferable to use a dose of 135 μg of G-CSF per kg body weight.

In one embodiment of the invention, a proportion of 20 to 50%, preferably a proportion of a third, of the total dose is to be given as a bolus at the start of treatment in the form of a rapid intravenous injection (e.g. within about 20 min), while the remaining proportion is to be administered via the intravenous route continuously over a period of 2 to 7 days, preferably over 3 days, to maintain a constantly high serum level.

According to the evaluation of the study described in Example 1, there is a similar relationship between the success of treatment and the total dose of G-CSF used for the administration of a total dose of G-CSF without taking into account the patient's weight. Alternatively, therefore, according to this invention a total dose of G-CSF of 2 to 16 mg (corresponding to the dose levels of 30 μg of G-CSF per kg body weight as a total dose to 180 μg of G-CSF per kg body weight as a total dose), preferably 6 to 12 mg (corresponding to the dose levels of 90 μg of G-CSF per kg body weight as a total dose to 135 μg of G-CSF per kg body weight as a total dose), particularly preferably 9.5 to 12 mg (corresponding to the dose levels of 135 μg of G-CSF per kg body weight as a total dose) is to be used for the treatment without taking into account the weight of the respective patient.

The total dose range of 30 to 180 μg of G-CSF per kg body weight over a period of 3 days used for the treatment of stroke patients within the framework of this invention (Example 1) is substantially higher than the doses of G-CSF used for the indications so far registered. Nevertheless, the treatment for stroke patients according to this invention was well tolerated and did not lead to any safety-relevant side effects.

Treatment with G-CSF as a promotor of neuroregeneration according to this invention permits a comparatively late start of treatment after the stroke, compared e.g. with tPA treatment based on the thrombolytic effect, which is only registered for a start of treatment up to 3 h after the stroke. Accordingly, the patients included in the study according to the invention had had strokes between 4 and 18 h before the start of treatment. On average, treatment was started about 10 h after the stroke.

The statistical evaluation of this study (Example 1) showed the administration of G-CSF according to the invention to display efficacy at an initial size (determined by DWI) of 16 cm³ or more.

In addition, it was found, surprisingly, that administration of G-CSF according to the invention is particularly suitable for the treatment of severe strokes which have a comparatively large infarct volume. Although the effect of the treatment according to the invention is only small in small infarcts, treatment of large infarcts according to the invention had a large effect. Accordingly, administration of G-CSF according to the invention should preferably be used to treat infarcts with an infarct volume of at least 16 cm³ (determined by DWI), and preferably at least 25 cm³ (determined by DWI, FIG. 3), and is particularly preferable for the treatment of infarcts with an infarct volume of at least 50 cm³ (determined by DWI, FIG. 2).

According to a preferred embodiment of the use in accordance with the invention, therefore, the the patients are tested before the treatment to determine whether they have a stroke with an initial infarct volume determined by DWI of the specified minimum volume (i.e. 16 cm³, 25 cm³, or 50 cm³).

The invention therefore also concerns a method for the identification of stroke patients who respond to treatment including the administration of G-CSF, including the steps
a) DWI-based determination of the infarct volume and
b) identification of patients with an infarct volume of at least 16 cm³ as candidates for the said treatment.

In addition, the invention therefore also concerns a method for the identification of stroke patients who respond to a treatment including the administration of G-CSF, including the steps
a) DWI-based determination of the infarct volume and
b) identification of patients with an infarct volume with involvement of the cortical cerebral tissue as candidates for the said treatment.

This method in accordance with the invention is particularly useful for the identification of patients who respond particularly well to the use of G-CSF in accordance with the invention. It can, however, also be used in general to identify patients who in general respond particularly well to G-CSF treatment.

According to a preferred embodiment of the invention, administration of G-CSF is carried out as defined within the framework of the use in accordance with the invention.

While the small infarcts in this study are predominantly in subcortical locations, the larger infarcts generally involved cortical brain tissue. The larger the infarct, the more severe the damage to cortical brain tissue. Thus, it is to be assumed that the cortical brain tissue is particularly responsive to treatment with G-CSF. Accordingly, administration of G-CSF according to the invention should preferably be used in the treatment of infarcts involving cortical brain tissue.

The active ingredient G-CSF can be formulated for administration in accordance with the invention with one or more pharmaceutically tolerable excipients. The term “pharmaceutically tolerable” relates to molecules and compositions which are physiologically tolerated and which do not typically cause allergies or adverse reactions such as spells of dizziness.

The term “excipient” means a diluent, adjuvant, vehicle or other excipient with which the active ingredient is to be administered. Such pharmaceutical excipients may be sterile liquids such as water, saline solutions, buffer solutions, glucose solutions, glycerol solutions, detergent solutions, DMSO or water and oil emulsions. Water, saline solutions, buffer solutions, glucose solutions and glycerol solutions are preferably used as excipients, particularly for solutions of the active ingredient for injection. Particularly preferable is the use of G-CSF as an active ingredient in combination with the excipients sodium acetate buffer with a pH of 4, sorbitol and the detergent Tween 80, as well as a glucose solution.

The term “treatment” means the slowing down, interruption, arrest, reversal or stoppage of the progression of the state after the stroke, which does not necessarily require the complete elimination of all the signs and symptoms of stroke. Furthermore, it is not necessary for the treatment to show effectiveness in 100% of the patients treated, rather, the term “treatment” is intended to mean that a statistically significant proportion of patients can be treated effectively, in such a way that the symptoms and clinical signs show at least an improvement. The person skilled in the art can easily establish whether the proportion is statistically significant using various statistical methods (e.g. confidence intervals, determination of them p value, Student's t-test, Mann-Whitney test etc.). Preferred confidence intervals have a confidence of at least 90%, at least 95%, at least 97%, at least 98% or at least 99%. The preferred p values are 0.1, 0.05, 0.01, 0.005 or 0.0001.

“Effect”, “effectiveness” or “efficacy” within the framework of this invention is taken to mean the extent of the treatment success, determined e.g. on the basis of the improvement in the clinical signs and symptoms. Suitable assessment criteria for such improvements within the framework of stroke treatment include, but are not limited to, the infarct size or neurological rating scales such as the NIH stroke scale or the modified Rankin scale.

In other embodiments, the stroke treatment according to the invention can be combined with the administration of one or more additional factors. “Additional factors” according to the invention means any substance which supports the effect of the treatment of stroke with G-CSF according to the invention. Suitable additional factors include e.g. factors with a neuroprotective effect such as erythropoietin, BDNF, VEGF, CNTF, GM-CSF or inflammation-modulating factors. The additional administration of bradykinin or analogous substances in intravenous administration can support the transport of the active substances to the brain (Emerich et al., Clin Pharmacokinet 2001; 40: 105-123; Siegal et al., Clin Pharmacokinet 2002; 41: 171-186). Antiapoptotic agents or agents which assist passage across the blood-brain barrier can also be used. The administration of additional factors can take place at the same time as, before, or after administration of G-CSF according to the invention.

The invention is also explained in more detail in the figures.

The accompanying figures show:

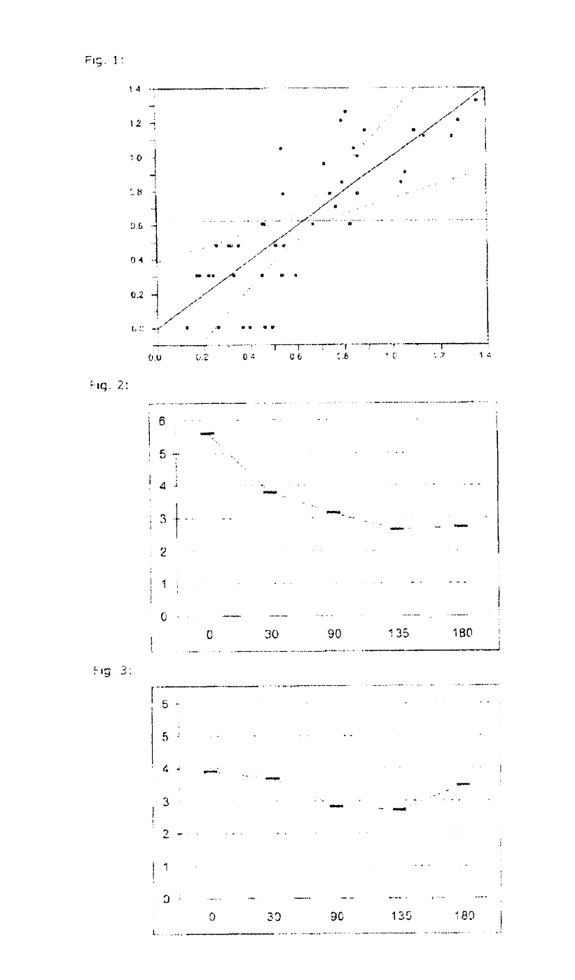

FIG. 1

Correlation for the linear model described in Example 1.

The response variable is plotted for the individual patients as a function of the parameters which are included in the model. The coefficient of correlation for the model is 0.67 and the p value for the model is less than 0.0001.

FIG. 2

Dose-effect curve for large infarcts.

The success of treatment estimated from the statistical model in Example 1 (given as the value of the NIH stroke scale 90 days after the start of treatment) is plotted as a function of the G-CSF dose level to be administered (in μg per kg body weight, where “0” corresponds to treatment with placebo). The dose-effect curve is derived from the statistical model for patients with an initial value of 8.65 in the NIH stroke scale, an age of about 70 years and a baseline infarct volume (determined by DWI) of about 50 cm³.

FIG. 3

Dose-effect curve for medium sized infarcts.

The success of treatment estimated from the statistical model in Example 1 (given as the value of the NIH stroke scale 90 days after the start of treatment) is plotted as a function of the G-CSF dose level to be administered (in μg per kg body weight, where “0” corresponds to treatment with placebo). The dose-effect curve is derived from the statistical model for patients with an initial value of 8.65 in the NIH stroke scale, an age of about 70 years and a baseline infarct volume (determined by DWI) of about 25 cm³.

### DESCRIPTION OF THE EXEMPLARY EMBODIMENT

### Example 1

The safety and efficacy of the use of G-CSF for the treatment of cerebral stroke was investigated in a placebo-controlled double-blind study with escalating dose steps under the conditions laid down by the Ethics Committee. A total of 43 patients were included in the study. 14 patients were given placebo (group P), while sets of 7 patients each were given a total dose of G-CSF of 30 μg per kg body weight (group I), 90 μg per kg body weight (group II) or 180 μg per kg body weight (group IV). 8 patients were given a total dose of G-CSF of 135 μg per kg body weight (group III).

Male and female patients aged 40 to 87 years with acute cerebral stroke about 4 to 18 h before the start of treatment were included. On average, treatment was started 10 h after the infarct. The patients included in the study had an baseline infarct size determined by DWI of between about 1 and 100 cm³ and an initial rating on the NIH stroke scale between 1 and 19. Another inclusion criterion was that the baseline infarct area determined by PWI had to be larger than the baseline infarct area determined by DWI (DWI/PWI mismatch). The patients were given placebo or the active ingredient intravenously over a period of 3 days from the start of treatment, one-third of the total dose being given at the start of treatment, as a bolus infusion over about 20 min. The remaining two-thirds of the total dose were then administered as a steady infusion over the entire treatment period, to ensure a constantly high serum level. The active ingredient used was recombinant G-CSF (Neupogen®) in the appropriate standard buffer (10 mM sodium acetate buffer with a pH of 4, 50 mg/ml of sorbitol and 0.004% Tween 80), which was diluted in glucose solution for infusion.

At none of the dose levels used were any safety-relevant side effects observed in the stroke patients as a result of treatment with G-CSF according to the invention.

To assess the success of treatment, the neurological status of the stroke patients was assessed 90 days after the start of treatment, using the NIH stroke scale. Parameters evidently having a significant influence on the success of treatment were identified before unblinding the study. A linear model was created on the basis of the following parameters:
A: Age of the patient
N₀:Value on the NIH stroke scale at the start of treatment
V: Infarct size determined by DWI at the start of treatment on a logarithmic scale
D: Total dose of G-CSF administered

The interaction between the total dose of G-CSF administered (D) and the infarct size determined by DWI at the start of treatment on a logarithmic scale (V) was also included in the model as an additional parameter (D*V).

The logarithm of the value from the NIH stroke scale 90 days after the end of treatment was used as a measure of the success of treatment (response variable Y).

The linear model thus had the following form:

Y=a₁*D+a₂*V+a₃*D*V+a₄*A+a₅*N₀+ε

where ε is the residual error term.

After unblinding, the parameters a₁ to a₅ were determined from the patient data with the aid of suitable statistical methods.

FIG. 1 shows the correlation between the input parameters and the results parameter according to the linear model obtained. The linear model displays very good correlation, with a coefficient of correlation r²=0.67 and a p value of <0.0001.

On the basis of this model, it is possible to estimate the expected treatment outcome (in the form of the NIH stroke scale value after 90 days) for treatments with placebo or the various G-CSF dose levels as a function of the patient's age, neurological status at the start of treatment (NIH stroke scale) and baseline infarct size (determined by DWI). Although the patient's age and initial neurological status have a significant influence on the general prognosis (younger patients and those with only mild initial neurological deficits can also be expected to show a generally good improvement in the neurological symptoms over the observation period of 90 days), they have no influence on the basic form of the dose-effect curve, i.e. on the difference of treatment outcome between placebo and G-CSF treatment in accordance with the invention, in this statistical model. On the other hand, according to this statistical model, the baseline infarct size (determined by DWI) does have a clear effect on the extent of the success of G-CSF treatment compared with placebo treatment. On the basis of the statistical model, administration of G-CSF in accordance with the invention is effective in infarcts with an initial size of 16 cm³ or more. Furthermore, on the basis of the statistical model it is possible to estimate that the treatment with G-CSF according to the invention (particularly G-CSF at a total dose of 135 μg per kg body weight) can be expected to produce a particularly clear improvement in neurological symptoms compared with placebo treatment (assessed on the basis of the NIH stroke scale after 90 days) in patients with a relatively large infarct volume of about 50 cm³ or more (FIG. 2). For patients with a moderate infarct size of about 25 cm³, a distinctly better neuronal symptom pattern can still be expected after G-CSF treatment according to the invention compared with placebo treatment (FIG. 3). According to this statistical analysis, only a minor treatment success can be expected after G-CSF treatment of smaller infarcts.

The dose-effect curves estimated from the statistical model (FIG. 2 and FIG. 3) show that the optimal total dose for G-CSF treatment according to the invention can be expected to be about 135 μg per kg body weight. A further increase in the total dose of G-CSF cannot be expected to produce any further improvement in the success of treatment.

1-17. (canceled)

18. A method for treating a human patient with cerebral stroke, comprising administrating G-CSF at a total dose of 80 to 150 μg per kg body weight over a treatment period of 2 to 7 days.

19. The method as claimed in claim 18, wherein the total dose administered is between 120 and 150 μg per kg body weight.

20. The method as claimed in claim 18, wherein the total dose is administered over a treatment period of 3 days.

21. The method as claimed in claims 18, wherein the total dose is administered intravenously.

22. The method as claimed in claim 21, wherein a proportion of 20 to 50% of the total dose is to given as a bolus at the start of treatment and the remaining proportion is administered continuously over the treatment period.

23. The method as claimed in claim 22, wherein the proportion administered as a bolus is one-third of the total dose.

24. The method as claimed in claim 18, wherein the human patient has an infarct with cortical involvement.

25. The method as claimed in claim 24, further comprising prior to administering, testing the human patient to determine whether the human patient has an infarct with cortical involvement.

26. The method as claimed in claim 24, wherein the human patient has a stroke with a baseline infarct volume determined by DWI of at least 16 cm³.

27. The method as claimed in claim 26, wherein the human patient has a stroke with a baseline infarct volume determined by DWI of at least 25 cm³.

28. The method as claimed in claim 27, wherein the human patient has a stroke with a baseline infarct volume determined by DWI of at least 50 cm³.

29. The method as claimed in claim 26, further comprising prior to administering, testing the human patient to determine whether the human patient has a stroke with an initial infarct volume determined by DWI of at least the particular minimum volume.

30. A method for identifying a candidate stroke patient who can respond to G-CSF treatment, the method comprising
a. determining a DWI-based infarct volume in the stroke patient, and
b. identifying the stroke patient with an infarct volume of at least 16 cm³ as a candidate for said treatment.

31. A method for identifying a candidate stroke patient who responds to G-CSF treatment, the method comprising
a. determining DWI-based infarct volume in the stroke patient, and
b. identifying a patient with an infarct volume with involvement of cortical brain tissue as a candidate for said treatment.
